(19) United States
(12) Patent Application Publication
(10) Pub. No.: US 2009/0131450 A1
(43) Pub. Date: May 21, 2009

(54) USE OF PKC INHIBITORS IN DIABETIC COMPLICATIONS

(76) Inventors: Jurgen Wagner, Bottmingen (CH); Jean-Pierre Evenou, St. Louis (FR)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 12/305,249

(22) PCT Filed: Jun. 28, 2007
(86) PCT No.: PCT/EP2007/005746

§ 371 Date: Dec. 17, 2008

(30) Foreign Application Priority Data
Jun. 30, 2006 (GB) 0613162.7

(51) Int. Cl. A61K 31/506; C07D 403/14; A61P 3/10; A61K 31/4725

(52) U.S. Cl. 514/252.17; 544/284; 546/15; 514/278

(57) ABSTRACT

The present invention pertains to the use of a PKC inhibitor in the manufacture of a medicament in the treatment or prevention of diabetic complications.

The present invention relates to a new use of a PKC inhibitor in the treatment or prevention of diabetic disorders or complications such as nephropathy and cardiomyopathy.

Cardiac and renal complications of diabetes are a major cause of mortality and morbidity, however the basic cellular events that promote their progression remain elusive. It is likely that the initial lesion is induced by metabolic effects such as hyperglycaemia leading to adaptive changes in haemodynamics with enhanced production of growth factors and proinflammatory mediators, leading to inflammation, hypoxia and increased extracellular matrix (ECM) deposition.

Diabetic nephropathy is characterized by progressive glomerulosclerosis and tubulointerstitial fibrosis which are accompanied by proteinuria and a decline in GFR, ultimately leading to end-staged renal failure. Diabetic cardiomyopathy is characterized in its early stages by abnormal diastolic function, along with subtle abnormalities in systolic function, as demonstrated by reduced longitudinal fibre function. Histological studies of human diabetic hearts show increased extracellular matrix deposition, predominantly fibrillar collagens along with myocardial hypertrophy.

In spite of numerous treatment options for treating or preventing diabetic and its complications, nephropathy and cardiomyopathy continue to progress in the majority of diabetic patients and there remains a need for effective and safe treatment of these complications.

In accordance with the present invention, it has now surprisingly been found that PKC inhibitors which are described hereinbelow, can be used to prevent or treat diabetic disorders or complications, in particular disorders or complications caused by diabetes mellitus, such as diabetic nephropathy and cardiomypathy.

The present invention provides the use of PKC inhibitors in preventing, treating or delaying diabetic disorders or complications, such as nephropathy, cardiomyopathy or neuropathy, wherein the PKC inhibitors are selected from 3-(1.H.-indol-3-yl)-4-[2-(4-methyl-piperazin-1-yl)quinazolin-4-yl]-pyrrole-2,5-dione (referred to hereinafter as Compound A), 3-(1.H.-indol-3-yl)-4-[2-(piperazin-1-yl)-quinazolin-4-yl]-pyrrole-2,5-dione (referred to hereinafter as Compound B), and 3-[3-(4,7-Diaza-spiro[2,5]oct-7-yl)-isoquinolin-1-yl]-4-(7-methyl-1H-indol-3-yl)-pyrrole-2,5-dione (Compound C).

According to the invention, Compounds A, B and C are in free form or in a pharmaceutically acceptable salt form.

The most preferred compound is Compound A, even more preferred is the acetate salt thereof.

The compounds A, B and C are known and may be prepared as disclosed in the art, e.g. as described in U.S. Pat. No. 6,645,970 or EP1490355A1. The salts of compound A, compound B and compound C may be prepared in conventional manner and exhibit the same order of activity as the free compounds.

In a series of further specific or alternative embodiments, the present invention also provides:
1. A method for treating, preventing or delaying diabetic complications as described hereinbelow, said method comprising administering to an affected individual a therapeutically effective amount of Compound A, Compound B, Compound C or a salt thereof, more preferably Compound A or the acetate salt thereof.

A “diabetic complications” as defined in this application comprises, but is not limited to hyperglycemia, hyperinsulinaemia, insulin resistance, impaired glucose metabolism, conditions of impaired glucose tolerance (IGT), conditions of impaired fasting plasma glucose, obesity, diabetic nephropathy, glomerulosclerosis, diabetic neuropathy, diabetic cardiomyopathy and syndrome X, and end-stage renal disease.

Preferably, the present invention provides a method for treating, preventing or delaying diabetic complications comprising administering to an affected individual a therapeutically effective amount of Compound A or a salt thereof, for example the acetate salt thereof, wherein the diabetic complications is as hereinabove defined.

For example there is provided a method for treating, preventing or delaying diabetic nephropathy, diabetic cardiomyopathy or diabetic neuropathy, preferably diabetic nephropathy, comprising administering to an affected individual a therapeutically effective amount of Compound A, Compound B or a salt thereof, for example Compound A or a salt thereof, for example the acetate salt of Compound A.

In another embodiment of the invention, there is provided a method for treating, preventing or delaying hyperglycemia, hyperinsulinaemia, insulin resistance, glomerulosclerosis, diabetic cardiomyopathy and end-stage renal disease, comprising administering to an affected individual a therapeutically effective amount of Compound C or a salt thereof, for example the acetate salt thereof.

In yet another aspect the present invention provides:
2. Compound A, Compound B, Compound C or a pharmaceutically acceptable salt thereof, for use in a method as defined under 1 above;
3. Compound A, Compound B, Compound C or a pharmaceutically acceptable salt thereof, for use in the preparation of a pharmaceutical composition for use in a method as defined under 1 above;
4. A pharmaceutical composition for use in a method as defined under 1 above comprising Compound A, Compound B, Compound C or a pharmaceutically acceptable salt thereof, together with one or more pharmaceutically acceptable diluents or carriers therefor.

In a preferred embodiment of the invention, there is provided the use of Compound A in free form or in a pharmaceutically acceptable salt form, e.g. the acetate salt thereof, in treating or preventing a diabetic complication as hereinabove defined, for example diabetic nephropathy, diabetic neuropathy or diabetic cardiomyopathy, preferably diabetic nephropathy.

In another preferred embodiment of the invention, there is provided the use of Compound C in free form or in a pharmaceutically acceptable salt form, e.g. the acetate salt thereof, for treating or preventing diabetic cardiomyopathy.

Utility of compound A, B or C, in the treatment of diabetic complications, as hereinabove specified, may be demonstrated in animal test methods as well as in clinic, for example in accordance with the methods hereinafter described.

A Binding affinity of Compound A, B and C to individual human PKC receptors may be determined in Protein Kinase C assay. Such an assay can be done according to published methods (e.g. D. Geiges et al. Biochem. Pharmacol. 1997; 53:865-875), e.g. as disclosed in EP1337527A1, the content regarding the Protein Kinase C assay being incorporated herein by reference.

B In vivo

Ren-2 rats (e.g. as disclosed in D. J. Kelly et al, Kidney Int, Vol 54, 1998, p343-352) The effect of Compound A, B or C, on diabetic complications is tested in transgenic (mRen-2)27 rats (also called “Ren-2 rats”), model that develops hypertension and many of the structural and functional characteristics of human diabetic nephropathy when diabetes is induced by streptozotocin, resulting in a progressive disease with many of the structural, functional and molecular characteristics of human diabetic nephropathy. The progression of this disease is associated with an increase in renal and cardiac growth factors and extra cellular matrix accumulation.

The effect of the compounds of the invention on diabetic nephropathy may be studied with the Ren-2 rats model, as disclosed in D. J. Kelly et al, Diabetes, Vol 52, February 2003, p512-518: Diabetes is induced by injection of streptozotocin, 55 mg/kg. All diabetic rats receive insulin (human isophane) 2 units/day to promote body weight and reduce mortality. The experimental period continues for 16 weeks in Ren-2 rats to study advanced renal and cardiac disease. Rats are randomised to the following groups:

Group 1: non-diabetic Ren-2, no drug
Group 2: non-diabetic Ren2, Compound C
Group 3: non-diabetic Ren-2, Compound A
Group 4: diabetic Ren-2
Group 5: diabetic Ren-2, drug Compound C, dose 1
Group 6: diabetic Ren-2, drug Compound A, dose 1
Group 7: diabetic Ren-2, drug Compound C, dose 2
Group 8: diabetic Ren-2, drug Compound A, dose 2
N=20 per group (160 rats)

### a) Clinical Parameters

Serial measurements of blood pressure (4, 8, 12, 16 weeks), glycaemic control (weekly), HbA1c (end point) and clinical parameters are undertaken at intervals as per our standard protocols for animal studies.

### b) Renal Function (Primary Endpoints)

Serial measurements of renal function are made by the measurement of plasma creatinine and urea (autoanalyser), albuminuria (radioimmunoassay) and GFR (single shot isotopic technique) as per standard protocols for animal studies.

### c) Renal Structure (Secondary Endpoints)

At sacrifice (16 weeks of diabetes) the left kidney is fixed in formalin. The following morphological measurements is performed:

Three micron-paraffin sections stained with periodic acid Schiffs, haematoxylin and eosin and Masson trichrome are used for Light microscopy (LM) to measure glomerular volume, glomerulosclerotic and tubulointerstitial injury indices and cardiac hypertrophy. Immunohistochemistry using the avidin-biotin methods is used to examine:

1. Type I, III and IV collagen to assess the extent of matrix deposition,
2. TGF-β and phospho-Smad 2 to assess the modulation of profibrotic growth factors and their activity.

The spatial distribution and quantitation of the immunohistochemistry by image analysis provide specific information regarding the cell type and location of disease progression. Immunolocalisation for TUNEL (apoptosis) and PCNA (Proliferation) are used to determine the state of cell cycle.

### d) Molecular Biology

The right kidney is snap frozen in liquid nitrogen and used for molecular biological experiments. Tissue and cell specific changes in expression may be quantified using immunohistochemistry with quantitative image analysis and real time PCR (ABI Prism 7700, Perkin Elmer Biosystems, Foster City, Calif.) with TaqMan, Such a PCR analysis is a technique well known by the one skilled in the art.

When administered i.v. at a dose of from 0.1 to 10 mg/kg, Compound A, Compound B or Compound C, have a beneficial effect on the diabetic complications.

C Clinical Trial: Clinical Double-Blind, Randomized, Parallel-Group Study in Subjects with Type 2 Diabetes Mellitus Inadequately Controlled on Diet Alone.

Subjects with a diagnosis of type 2 diabetes mellitus who have not achieved near normoglycemia (HbA_{1c}<6.8%) on diet only are chosen for this trial. The effects on glycemic control achieved with Compound A, B and C are determined in this study after 24 weeks with the control achieved on placebo, all subjects continuing with the same diet as in the period before treatment. Measures of glycemic control are validated surrogate endpoints for the treatment of diabetes. HbA_{1c} is the single most reliable measurement for assessing glycemic control (D. Goldstein et al, Tests of Glycemia in Diabetes; Diabetes Care 1995, 18(6), 896-909) and is the primary response variable in this study. Since glycosylation of hemoglobin is determined by the glucose concentration at the time each red blood cell is made, HbA_{1c} provides an estimate of mean blood glucose for the previous three months.

Before starting with the double-blind treatment for 24 weeks, the subjects are administered for four weeks the placebos before breakfast, lunch and dinner (period I).

The subjects are then separated into four treatment groups for the 24-week double-blind study (period II) as depicted in Table below:

placebo**
placebo*
Compound A 10 mg*
Compound A 10 mg**
*administered before breakfast, lunch, and dinner;
**administered once daily with breakfast

Approximately 100 subjects are randomized per treatment group. The total study duration including the run-in period for each subject is 28 weeks. Statistical analysis can be carried out by methods known in the art.

The subject is advised not to take the morning dose of study medication or eat breakfast on the day of a scheduled study visit. The morning dose is administered by site personnel after the collection of all fasting laboratory samples and completion of all study procedures. Visits are scheduled to be performed at 2 week intervals during period I, and 4 to 8 week intervals during period II. Subjects have fasted for at least 7 hours at the time of each visit. All blood samples for laboratory evaluations are drawn between 7:00 AM and 10:00 AM. All tests are conducted in accordance with Good Laboratory Practice principles following procedures known in the art.

HbA_{1c} is measured by High Performance Liquid Chromatography (HPLC) using the ion-exchange method on a Bio-Rad Diamat analyzer. A back-up affinity method are used if hemoglobin variants or hemoglobin degradation peaks are observed.

Further parameters to be determined are fasting plasma glucose (FPG), fasting lipids (total, HDL (high density lipoprotein)- and LDL (low density lipoprotein)-cholesterol, and triglycerides) and body weight. FPG will be measured using the hexokinase method and LDL-cholesterol will be calculated using the Friedewald formula if triglycerides are <400 mg/dL (4.5 mmol/l).

Various parameters of the study described above can be modified, e.g. in order to optimize the dosage for special diseases or indications mentioned herein, to cope with tolerability problems during the study or to obtain similar or identical results with less efforts. For example, a different subject population can be involved in such a clinical trial, e.g. subjects with a diagnosis of type 2 diabetes mellitus who have achieved near normoglycemia (HbA_{1c}<6.8%) on diet alone, subjects with diseases other than diabetes mellitus, e.g. other metabolic disorders, or subjects selected by other criteria, such as age or sex; the subject number can be decreased, e.g. to a number of between 70 and 100, subjects per treatment group; the term of the placebo run-in period (period 1) can be changed, i.e. it can be extended, shortened or deleted; the visit schedule can be extended, e.g. to every 10, 12 or 14 weeks; the visit instructions can be changed, e.g. the instruction that blood samples for laboratory evaluations have to be drawn between 7:00 AM and 10:00 AM; HbA_{1c} can be determined by other means; or one or more of the parameters to be determined during the study mentioned above, e.g. FPG or fasting lipids, can be deleted or the determination of additional parameters (see below) can be added.

Additional parameters can be determined in the course of the study, e.g. by additional tests. Such additional tests can comprise the analysis of body liquids in order to determine amounts or numbers for parameters such as those listed below and can serve e.g. the purpose of determining the tolerability of the administered active ingredients: determination of hematocrit and hemogloblin, platelet count, erythrocyte count, total and differential leukocyte count (basophils, eosinophils, lymphocytes, monocytes, segmented neutrophils and total neutrophils); determination of albumin, alkaline phosphatase, alanine amino transferase (serum glutamic pyruvic transaminase), aspartate amino transferase (serum glutamic oxaloacetic transaminase), blood urea nitrogen or urea, bicarbonate, calcium, chloride, total creatine phosphokinase (CPK), creatine phosphokinase muscle-brain fraction isoenzyme (if CPK is elevated), direct bilirubin, creatinine, γ-glutamyl transferase, lactate dehydrogenase, potassium, sodium, total bilirubin, total protein and uric acid in the blood; determination of bilirubin, glucose, ketones, pH, protein, and specific gravity in the subjects urine; determination of body weight, blood pressure (systolic and diastolic, after 3 minutes sitting) and radial pulse (after 3 minutes sitting).

The results of the studies show that Compound A, B and C can be used for the prevention and preferably the treatment of type 2 diabetes mellitus and complications thereof.

According to the invention, Compounds A, B or C may be administered by any conventional route, in particular enterally, e.g. orally, e.g. in the form of tablets or capsules, or parenterally, e.g. in the form of injectable solutions or suspensions, topically, e.g. in the form of lotions or gels, or in a nasal or a suppository form.

Pharmaceutical compositions comprising Compound A, B or C in free form or in pharmaceutically acceptable salt form in association with at least one pharmaceutical acceptable carrier or diluent may be manufactured in conventional manner by mixing with a pharmaceutically acceptable carrier or diluent. Unit dosage forms for oral administration contain, for example, from about 0.1 mg to about 500 mg of active substance.

Daily dosages required in practicing the method of the present invention will vary depending upon, for example, the compound used, the host, the mode of administration, the severity of the condition to be treated. An indicated daily dosage for oral administration in the larger mammal, e.g. humans, is in the range from about 0.5 mg to about 2000 mg active ingredient, e.g. Compound A, B or C, conveniently administered, for example, in divided doses up to four times a day or in retard form.

Compound A, or B, preferably Compound A, or a pharmaceutically acceptable salt thereof, may be administered as the sole ingredient or together with at least one active ingredient selected from the group consisting of
(i) PPAR agonist;
(ii) HDL increasing compounds;
(iii) anti-diabetics as hereinbelow defined;
(iv) an anti-hypertensive agent;
(v) cholesterol absorption modulator;
(vi) apo-A1 analogs and mimetics;
(vii) renin inhibitors;
(viii) thrombin inhibitors;
(ix) aldosterone inhibitors;
(x) GLP-1 agonists;
(xi) glucagon receptor antagonists;
(xii) cannabinoid receptor 1 antagonists;
(xiii) anti-obesity agents;
(xiv) inhibitors of platelet aggregation;
(xv) DPP-IV inhibitors; and
(xvi) prosclerotic growth factors
or, in each case, a pharmaceutically acceptable salt thereof;
   and optionally a pharmaceutically acceptable carrier.

In a preferred embodiment of the invention, Compound C, or a pharmaceutically acceptable salt thereof, is administered together with at least one active ingredient selected from the group consisting of;
(i) HDL increasing compounds;
(ii) an anti-hypertensive agent;
(iii) cholesterol absorption modulator;
(iv) apo-A1 analogs and mimetics;
(v) renin inhibitors;
(vi) thrombin inhibitors;
(vii) aldosterone inhibitors;
(viii) glucagon receptor antagonists;
(ix) cannabinoid receptor 1 antagonists;
(x) anti-obesity agents;
(xi) inhibitors of platelet aggregation;
(xii) DPP-IV inhibitors; and
(xiii) prosclerotic growth factors
or, in each case, a pharmaceutically acceptable salt thereof;
   and optionally a pharmaceutically acceptable carrier.

PPAR agonists are meant to include but not be limited to selective PPAR alpha agonists, PPAR gamma agonists or PPAR delta agonists and dual alpha/gamma agonists and dual alpha/delta agonists.

Selective PPAR alpha agonists include those disclosed in international patent application US2005132224 published on Mar. 16, 2006 with publication No. WO 06/029349.

Dual acting PPAR alpha/gamma agonists include those disclosed in international patent application PCT/EP02/13025 published on May 30, 2003 with publication No. WO 03/043985, particularly compound 19 of Example 4, shown as compound 4-19.

Anti-diabetics include PPAR delta compounds; insulin sensitivity enhancers which restore impaired insulin receptor function to reduce insulin resistance and consequently enhance the insulin sensitivity.

Examples of PPAR delta compounds include the compounds of formula

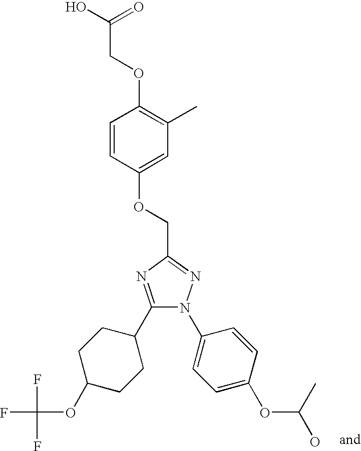

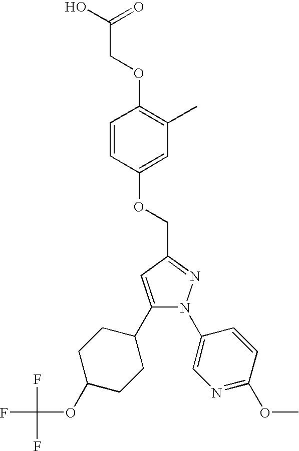

An appropriate insulin sensitivity enhancer is, for example, an appropriate hypoglycemic thiazolidinedione derivative (glitazone).

An appropriate glitazone is, for example, (S)-((3,4-dihydro-2-(phenyl-methyl)-2H-1-benzopyran-6-yl)methyl-thiazolidine-2,4-dione (englitazone), 5-{[4-(3-(5-methyl-2-phenyl-4-oxazolyl)-1-oxopropyl)-phenyl]-methyl}-thiazolidine-2,4-dione (darglitazone), 5-{[4-(1-methyl-cyclohexyl)methoxy)-phenyl]methyl}thiazolidine-2,4-dione (ciglitazone), 5-{[4-(2-(1-indolyl)ethoxy)phenyl]methyl}thiazolidine-2,4-dione (DRF2189), 5-{4[2-(5-methyl-2-phenyl-4-oxazolyl)-ethoxy)]benzyl}thiazolidine-2,4-dione (BM-13.1246), 5-(2-naphthylsulfonyl)-thiazolidine-2,4-dione (AY-31637), bis{4-[(2,4-dioxo-5-thiazolidinyl)methyl]phenyl}methane (YM268), 5-{4-[2-(5-methyl-2-phenyl-4-oxazoly)-2-hydroxyethoxy]benzyl}-thiazolidine-2,4-dione (AD-5075), 5-[4-(1-phenyl-1-cyclopropanecarbonylamino)-benzyl]-thiazolidine-2,4-dione (DN-108) 5-{[4-(2-(2,3-dihydroindol-1-yl)ethoxy)phenyl]methyl}thiazolidine-2,4-dione, 5-[3-(4-chloro-phenyl])-2-propynyl]-5-phenylsulfonyl)thiazolidine-2,4-dione, 5-[3-(4-chlorophenyl])-2-propynyl]-5-(4-fluorophenyl-sulfonyl)thiazolidine-2,4-dione, 5-{[4-(2-(methyl-2-pyridinyl-amino)-ethoxy)phenyl]methyl}thiazolidine-2,4-dione (rosiglitazone), 5-{[4-(2-(5-ethyl-2-pyridyl)ethoxy)phenyl]-methyl}-thiazolidine-2,4-dione (pioglitazone), 5-{[4-((3,4-dihydro-6-hydroxy-2,5,7,8-tetramethyl-2H-1-benzopyran-2-yl)methoxy)-phenyl]-methyl}-thiazolidine-2,4-dione (troglitazone), 5-[6-(2-fluoro-benzyloxy)naphthalen-2-ylmethyl]-thiazolidine-2,4-dione (MCC555), 5-{[2-(2-naphthyl)-benzoxazol-5-yl]-methyl}thiazolidine-2,4-dione (T-174) and 5-(2,4-dioxothiazolidin-5-ylmethyl)-2-methoxy-N-(4-trifluoromethyl-benzyl)benzamide (KRP297). Preferred are pioglitazone, rosiglitazone and troglitazone. Anti-diabetics include non-glitazone type PPARγ agonists, especially N-(2-benzoylphenyl)-L-tyrosine analogues, e.g. GI-262570, and JTT501.

Anti-hypertensive agents include angiotensin converting enzyme inhibitors (ACE-inhibitors); renin inhibitors, calcium channel blockers, diuretics, beta-blockers, neutral endo-peptidase inhibitors (NEP inhibitors), endothelin converting enzyme inhibitors (ECE inhibitors) and angiotensin II (AT₁) receptor antagonists, optionally in combination with a diuretic, for example, Co-Diovan®. The interruption of the enzymatic degradation of angiotensin I to angiotensin II with ACE-inhibitors is a successful variant for the regulation of blood pressure and thus also makes available a therapeutic method for the treatment of congestive heart failure.

The class of ACE inhibitors comprises compounds having differing structural features. For example, mention may be made of the compounds which are selected from the group consisting alacepril (cf. EP 7477), benazepril (cf. EP 72352), benazeprilat (cf. EP 72352), captopril (cf. U.S. Pat. No. 4,105,776), ceronapril (cf. EP 229520), cilazapril (cf. EP 94095), delapril (cf. EP 51391), enalapril (cf. EP 12401), enaprilat (cf. EP 12401), fosinopril (cf. EP 53902), imidapril (cf. EP 95163), lisinopril (cf. EP 12401), moveltipril (cf. ZA 82/3779), perindopril (cf. EP 49658), quinapril (cf. EP 49605), ramipril (cf. EP 79022), spirapril (cf. EP 50800), temocapril (cf. EP 161801), trandolapril (cf. EP 551927), and moexipril, or, in each case, a pharmaceutically acceptable salt thereof.

Preferred ACE inhibitors are those agents that have been marketed, most preferred are benazepril and enalapril.

Comprised are likewise the corresponding stereoisomers as well as the corresponding crystal modifications, e.g. solvates and polymorphs, which are disclosed therein and, where applicable, all pharmaceutically acceptable salts thereof.

The compounds to be combined can be present as pharmaceutically acceptable salts. If these compounds have, for example, at least one basic center, they can form acid addition salts. Corresponding acid addition salts can also be formed having, if desired, an additionally present basic center. The compounds having an acid group (for example COOH) can also form salts with bases.

The class of AT₁ receptor antagonists comprises compounds having differing structural features, essentially preferred are the non-peptidic ones. For example, mention may be made of the compounds which are selected from the group consisting of valsartan (cf. EP 443983), losartan (cf. EP253310), candesartan (cf. 459136), eprosartan (cf. EP 403159), irbesartan (cf. EP454511), olmesartan (cf. EP 503785), tasosartan (cf. EP539086), telmisartan (cf. EP 522314), the compound with the designation E-4177 of the formula

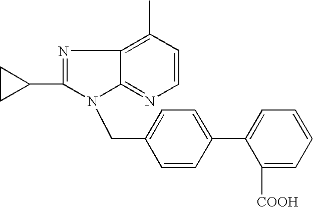

the compound with the designation SC-52458 of the following formula

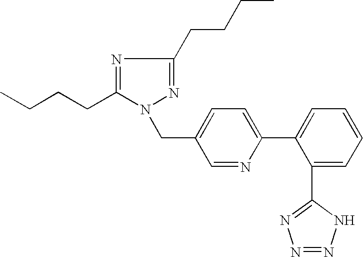

and the compound with the designation the compound ZD-8731 of the following formula

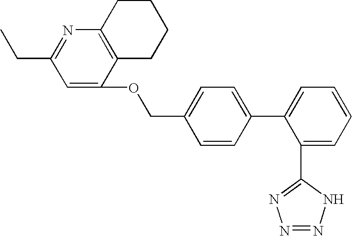

or, in each case, a pharmaceutically acceptable salt thereof.

Preferred AT₁-receptor antagonist are those agents which have been marketed, most preferred is Diovan® and Co-Diovan® or a pharmaceutically acceptable salt thereof.

The class of CCBs essentially comprises dihydropyridines (DHPs) and non-DHPs, such as diltiazem-type and verapamil-type CCBs.

A CCB useful in said combination is preferably a DHP representative selected from the group consisting of amlodipine, felodipine, ryosidine, isradipine, lacidipine, nicardipine, nifedipine, niguldipine, niludipine, nimodipine, nisoldipine, nitrendipine and nivaldipine, and is preferably a non-DHP representative selected from the group consisting of flunarizine, prenylamine, diltiazem, fendiline, gallopamil, mibefradil, anipamil, tiapamil and verapamil, and in each case, a pharmaceutically acceptable salt thereof. All these CCBs are therapeutically used, e.g., as anti-hypertensive, anti-angina pectoris or anti-arrhythmic drugs.

Preferred CCBs comprise amlodipine, diltiazem, isradipine, nicardipine, nifedipine, nimodipine, nisoldipine, nitrendipine and verapamil, or, e.g., dependent on the specific CCB, a pharmaceutically acceptable salt thereof. Especially preferred as DHP is amlodipine or a pharmaceutically acceptable salt, especially the besylate, thereof. An especially preferred representative of non-DHPs is verapamil or a pharmaceutically acceptable salt, especially the hydrochloride, thereof.

A diuretic is, e.g., a thiazide derivative selected from the group consisting of chlorothiazide, hydrochlorothiazide, methylclothiazide, amiloride, triamterene and chlorothalidon. The most preferred is hydrochlorothiazide.

Beta-blockers suitable for use in the present invention include beta-adrenergic blocking agents (beta-blockers) which compete with epinephrine for beta-adrenergic receptors and interfere with the action of epinephrine. Preferably, the beta-blockers are selective for the beta-adrenergic receptor as compared to the alpha-adrenergic receptors, and so do not have a significant alpha-blocking effect. Suitable beta-blockers include compounds selected from acebutolol, atenolol, betaxolol, bisoprolol, carteolol, carvedilol, esmolol, labetalol, metoprolol, nadolol, oxprenolol, penbutolol, pindolol, propranolol, sotalol and timolol. Where the beta-blocker is an acid or base or otherwise capable of forming pharmaceutically acceptable salts or prodrugs, these forms are considered to be encompassed herein, and it is understood that the compounds may be administered in free form or in the form of a pharmaceutically acceptable salt or a prodrug, such as a physiologically hydrolizable and acceptable ester. For example, metoprolol is suitably administered as its tartrate salt, propranolol is suitably administered as the hydrochloride salt, and so forth.

NEP inhibitors within the scope of the present invention include compounds disclosed in U.S. Pat. Nos. 5,223,516 and 4,610,816, herein incorporated by reference, including in particular N—[N-[1(S)-carboxyl-3-phenylproplyl]-(S)-phenylalanyl]-(S)-isoserine and N—[N-[((1S)-carboxy-2-phenyl)ethyl]-(S)-phenylalanyl]-β-alanine; compounds disclosed in U.S. Pat. No. 4,929,641, in particular N-[2(S)-mercaptomethyl-3-(2-methylphenyl)-propionyl]methionine; SQ 28603 (N-[2-(mercaptomethyl)-1-oxo-3-phenylpropyl]-β-alanine), disclosed in South African Patent Application 84/0670; UK 69578 (cis-4-[[[1-[2-carboxy-3-(2-methoxyethoxy)propyl]-cyclopentyl]carbonyl]amino]-cyclohexanecarboxylic acid) and its active enantiomer(s); thiorphan and its enantiomers; retro-thiorphan; phosphoramidon; and SQ 29072 (7-[[2-(mercaptomethyl)-1-oxo-3-phenylpropyl]amino]-heptanoic acid). Also suitable for use are any pro-drug forms of the above-listed NEP inhibitors, e.g., compounds in which one or more carboxylic acid groups are esterified.

NEP inhibitors within the scope of the present invention also include the compounds disclosed in U.S. Pat. No. 5,217,996, particularly, N-(3-carboxy-1-oxopropyl)-(4S)-p-phenylphenylmethyl)-4-amino-2R-methylbutanoic acid ethyl ester; the compounds disclosed in EP 00342850, particularly (S)-cis-4-[1-[2-(5-indanyloxycarbonyl)-3-(2-methoxyethoxy)propyl]-1-cyclopentanecarboxamido]-1-cyclohexanecarboxylic acid; the compounds disclosed in GB 02218983, particularly 3-(1-[6-endo-hydroxymethylbicyclo[2,2,1]heptane-2-exo-carbamoyl]cyclopentyl)-2-(2-methoxyethyl)propanoic acid; the compounds disclosed in WO 92/14706, particularly N-(1-(3-(N-t-butoxycarbonyl-(S)-prolylamino)-2(S)-t-butoxy-carbonylpropyl)cyclopentanecarbonyl)-O-benzyl-(S)-serine methyl ester; the compounds disclosed in EP 00343911; the compounds disclosed in JP 06234754; the compounds disclosed in EP 00361365, particularly 4-[([2-(Mercaptomethyl)-1-oxo-3-phenylpropyl]amino]benzoic acid; the compounds disclosed in WO 90/09374, particularly 3-[(1-(Cis-4-carboxycarbonyl-cis-3-butylcyclohexyl-r-1-carboamoyl)cyclopentyl]-2S-(2-methoxyethoxymethyl)propanoic acid; the compounds disclosed in JP 07157459, particularly N-((2S)-2-(4-biphenylmethyl)-4-carboxy-5-phenoxyvaleryl)glycine; the compounds disclosed in WO 94/15908 particularly N-(1-(N-hydroxycarbamoylmethyl)-1-cyclopentanecarbonyl)-L-phenylalanine; the compounds disclosed in U.S. Pat. No. 5,273,990 particularly (S)-(2-biphenyl-4-yl)-1-(1H-tetrazol-5-yl)ethylamino) methylphosphonic acid; the compounds disclosed in U.S. Pat. No. 5,294,632 particularly (S)-5-(N-(2-(phosphonomethylamino)-3-(4-biphenyl)propionyl)-2-aminoethyl)tetrazole; the compounds disclosed in U.S. Pat. No. 5,250,522, particularly β-Alanine, 3-[1,1′-biphenyl]-4-yl-N—[diphenoxyphosphinyl)methyl]-L-alanyl; the compounds disclosed in EP 00636621, particularly N-(2-carboxy-4-thienyl)-3-mercapto-2-benzylpropanamide; the compounds disclosed in WO 93/09101, particularly 2-(2-mercaptomethyl-3-phenylpropionamido)thiazol-4-ylcarboxylic acid; the compounds disclosed in EP 00590442 particularly ((L)-(1-((2,2-dimethyl-1,3-dioxolan-4-yl)-methoxy)carbonyl)-2-phenylethyl)-L-phenylalanyl)-β-alanine, N—[N-[(L)-[1-[(2,2-dimethyl-1,3-dioxolan-4-yl)-methoxy]carbonyl]-2-phenylethyl]-L-phenylalanyl]-(R)-alanine, N-1-[N-[(L)-1-carboxy-2-phenylethyl]-L-phenylalanyl]-(R)-alanine, N-[2-acetylthiomethyl-3-(2-methyl-phenyl)propionyl]-methionine ethyl ester, N-[2-mercaptomethyl-3-(2-methylphenyl)propioyl]-methionine, N-[2(S)-mercaptomethyl-3-(2-methylphenyl)propanoyl]-(S)-isoserine, N—(S)-[3-mercapto-2-(2-methylphenyl)propionyl]-(S)-2-methoxy-(R)-alanine, N-[1-[[1(S)-benzyloxycarbonyl-3-phenylpropyl]amino]cyclopentylcarbonyl]-(S)-isoserine, N-[1-[1-[(S)-carbonyl-3-phenylpropy]amino]-cyclopentylcarbonyl]-(S)-isoserine, 1,1′-[dithiobis-[2(S)-(2-methylbenzyl)-1-oxo-3,1-propanediyl]]-bis-(S)-isoserine, 1,1′-[dithiobis-[2(S)-(2-methylbenzyl)-1-oxo-3,1-propanediyl]]-bis-(S)-methionine, N-(3-phenyl-2-(mercaptomethyl)-propionyl)-(S)-4-(methylmercapto)methionine, N-[2-acetylthiomethyl-3-phenyl-propionyl]-3-aminobenzoic acid, N-[2-mercaptomethyl-3-phenyl-propionyl]-3-aminobenzoic acid, N-[1-(2-carboxy-4-phenylbutyl)-cyclopentanecarbonyl]-(S)-isoserine, N-[1-(acetylthiomethyl)cyclopentane-carbonyl]-(S)-methionine ethyl ester, 3(S)-[2-(acetylthiomethyl)-3-phenyl-propionyl]amimo-ε-caprolactam; and the compounds disclosed in WO 93/10773 particularly N-(2-acetylthiomethyl-3-(2-methylphenyl)propionyl)-methionine ethyl ester.

ECE inhibitors include SLV306.

Apo-A1 analogs and mimetics include the 18 amino acid D4F peptide as disclosed in Sequence ID No. 5 of U.S. Pat. No. 6,664,230 issued Dec. 16, 2003.

Renin inhibitors comprise, e.g., peptidic and, preferably, non-peptidic renin inhibitors.

A non-peptidic renin inhibitor is, e.g., ditekiren, terlakiren, zankiren, SPP-100 or a compound of formula

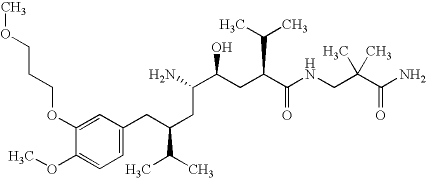

or, in each case, a pharmaceutically acceptable salt thereof.

The renin inhibitor of formula (I), chemically defined as 2(S),4(S),5(S),7(S)—N-(3-amino-2,2-dimethyl-3-oxopropyl)-2,7-di(1-methylethyl)+hydroxy-5-amino-8-[4-methoxy-3-(3-methoxy-propoxy)phenyl]-octanamide, is specifically disclosed in EP 678503A. Especially preferred is the hemi-fumarate salt thereof.

Non-peptidic renin inhibitor comprise those that are disclosed in WO 97/09311, especially corresponding renin inhibitors as disclosed in the claims and working examples, especially SPP100 of the formula

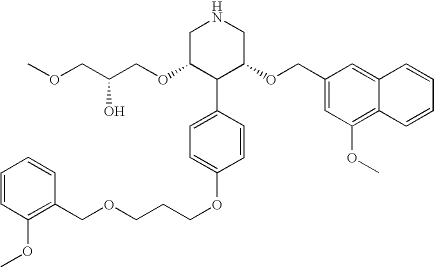

especially and of RO 66-1132 and RO-66-1168 of formula

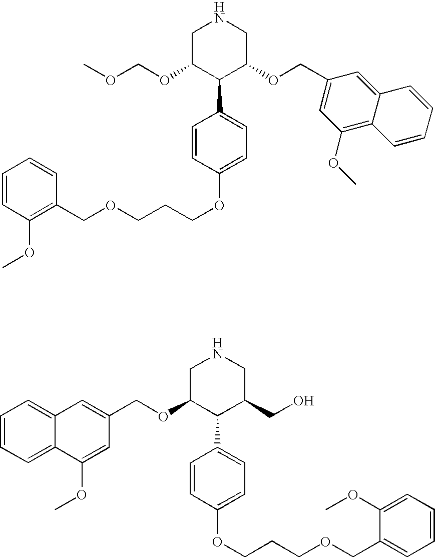

respectively, WO 04/002957, especially those renin inhibitors as disclosed in the working examples and claims. The corresponding subject matter of said WO applications is herein incorporated by reference into the present invention.

Cholesterol absorption modulators include Zetia® and KT6-971 (Kotobuki Pharmaceutical Co. Japan).

Thrombin inhibitors include Astra Zeneca's Ximelagatran (Exanta®) disclosed in WO 97/23499 published Oct. 12, 1999.

Aldosterone inhibitors include compounds having differing structural features. GLP-1 agonists includes GLP-1 analogs, GLP-1 receptor agonists and G-protein coupled receptor 120 (GPR120) agonists. GLP-1 analogs by way of example include Exendin-4™ (exenatide) or LY315902, Myers S R et al., Annual Meeting and Scientific Sessions of the American Diabetes Association, 1998, 58^{th}: Chicago (Abs 0748), and LY307161 Trautman, M., et al, Diabetologia, 2000, 43:Suppl1 (A146). GPR120 agonists include free fatty acids as set forth in Hirasawa, A. et al, Nature Medicine, Vol. 11, No. 1, January 2005.

Glucagon receptor antagonism includes administration of anti-sense molecules, for example RNA and oligonucleotides, to the gene encoding for the glucagon receptor and glucagon receptor antagonists such as, for example, small molecule antagonists which bind to the glucagon receptor and prevent or hinder the binding of natural ligands thereto. Anti-sense technology per se is known in the art. Disclosure of specific anti-sense oligonucleotides (ASOs) and methods used to identify ASOs are disclosed in Sloop, K., et al., The Journal of Clinical Investigation, Vol. 113, No. 11, June 2004, the disclosure of which is hereby incorporated by reference in its entirety as if set forth in full herein.

Cannabinoid receptor 1 (cb1) antagonists include, but are not limited to, compounds disclosed in international patent application US2005/32224 published on Mar. 16, 2006 with publication No. WO 06/029349 (compounds of Formulae Ia, Ib, Ic, Id, Ie, If, Ig and Ih).

Anti-obesity compounds, including Xenical®, Meridia® and cannabinoid receptor antagonists.

Inhibitors of platelet aggregation include Plavix®, aspirin and Clopidgrel®.

Prosclerotic growth factors include transforming growth factor-beta 1 (TGF beta).

HDL increasing compounds include but are not limited to cholesterol ester transfer protein inhibitors (CETP inhibitor). Examples of CETP inhibitors include JTT705 disclosed in example 26 of U.S. Pat. No. 6,426,365 issued Jul. 30, 2002 and pharmaceutically acceptable salts thereof.

The DPP-IV inhibitor can be peptidic or non-peptidic.

DPP-IV inhibitors are in each case generically and specifically disclosed in WO 98/19998, DE 196 16 486 A1, WO 00/34241, WO 95/15309, and WO01/52825 in each case in particular in the compound claims and the final products of the working examples, the subject-matter of the final products, the pharmaceutical preparations and the claims are hereby incorporated into the present application by reference to these publications. DPP728 and LAF237 are specifically disclosed in Example 3 of WO 98/19998 and Example 1 of WO 00/34241, respectively. A DPP-IV inhibitor is specifically described in Diabetes 1998, 47, 1253-1258.

The structure of the active agents identified by generic or tradenames may be taken from the actual edition of the standard compendium “The Merck Index” or the Physician's Desk Reference or from databases, e.g. Patents International (e.g. IMS World Publications) or Current Drugs. The corresponding content thereof is hereby incorporated by reference. Any person skilled in the art is fully enabled to identify the active agents and, based on these references, likewise enabled to manufacture and test the pharmaceutical indications and properties in standard test models, both in vitro and in vivo.

Any person skilled in the art is fully enabled to identify the active agents and, based on these references, likewise enabled to manufacture and test the pharmaceutical indications and properties in standard test models, both in vitro and in vivo.

Where compound A, B, C or salt thereof, is administered in conjunction with other drugs, dosages of the co-administered compound will of course vary depending on the type of co-drug employed, on the specific drug employed, on the condition to be treated, and so forth. The terms “co-administration” or “combined administration” or the like as utilized herein are meant to encompass administration of the selected therapeutic agents to a single patient, and are intended to include treatment regimens in which the agents are not necessarily administered by the same route of administration or at the same time.

In accordance with the foregoing the present invention provides in a yet further aspect:
5.1 A pharmaceutical combination comprising a) a first agent which is Compound A, or Compound B or a pharmaceutically acceptable salt thereof, preferably Compound A or the acetate salt thereof, and b) a co-agent, e.g. a second drug agent as defined above, for example a drug agent selected from the group consisting of an HDL increasing compound, a PPAR delta compound, a non-glitazone type PPARγ agonist, an anti-hypertensive agent, a cholesterol absorption modulator, an apo-A1 analog or mimetics, a renin inhibitor, a thrombin inhibitor, an aldosterone inhibitor, a GLP-1 agonist, a glucagon receptor antagonist, a cannabinoid receptor 1 antagonist, an anti-obesity agent, an inhibitor of platelet aggregation, a PPAR agonist, a prosclerotic growth factor, or a pharmaceutically acceptable salt thereof.
5.2 A pharmaceutical combination comprising a) a first agent which is Compound C or a pharmaceutically acceptable salt thereof, e.g. the acetate salt thereof, and b) a co-agent, e.g. a second drug agent as defined above, for example a drug agent selected from the group consisting of an HDL increasing compound, an anti-hypertensive agent, a cholesterol absorption modulator, an apo-A1 analog or mimetics, a renin inhibitor, a thrombin inhibitor, an aldosterone inhibitor, a glucagon receptor antagonist, a cannabinoid receptor 1 antagonist, an anti-obesity agent, an inhibitor of platelet aggregation, a prosclerotic growth factor, or a pharmaceutically acceptable salt thereof.
6. A method for treating, preventing or delaying diabetes or diabetic complications as described hereinbelow, for example for treating, preventing or delaying diabetic nephropathy, diabetic cardiomyopathy or diabetic neuropathy, comprising co-administration, e.g. concomitantly or in sequence, of a therapeutically effective amount of Compound A, B or C, or a pharmaceutically acceptable salt thereof, and a second drug substance, e.g. as indicated above.
7.1 A method for treating, preventing diabetes or diabetic complications, as hereinabove defined, or delaying their progression, said method comprising administering to an affected individual a therapeutically effective amount of a pharmaceutical combination comprising a) Compound A, B or C or a pharmaceutically acceptable salt thereof, preferably Compound A or a acetate salt thereof, and b) a co-agent selected from the group consisting of
   (i) PPAR agonist;
   (ii) HDL increasing compounds;
   (iii) anti-diabetics;
   (iv) an anti-hypertensive agent;
   (v) cholesterol absorption modulator;
   (vi) apo-A1 analogs and mimetics;
   (vii) renin inhibitors;
   (viii) thrombin inhibitors;
   (ix) aldosterone inhibitors;
   (x) GLP-1 agonists;
   (xi) glucagon receptor antagonists;
   (xii) cannabinoid receptor 1 antagonists;
   (xiii) anti-obesity agents;
   (xiv) inhibitors of platelet aggregation;
   (xv) DPP-IV inhibitors and
   (xvi) prosclerotic growth factors;
      or, in each case, a pharmaceutically acceptable salt thereof; and optionally a pharmaceutically acceptable carrier.
7.2 A method for treating, preventing diabetes or diabetic complications, as hereinabove defined, or delaying their progression, for example for treating, preventing or delaying diabetic nephropathy, diabetic cardiomyopathy or diabetic neuropathy, preferably diabetic nephropathy, said method comprising administering to an affected individual a therapeutically effective amount of a pharmaceutical combination as defined under 5.1. or 5.2.

The administration of a pharmaceutical combination of the invention results in a beneficial effect, especially a synergistic effect. For example combined treatment can result in surprising prolongation of efficacy, less side-effects, lower doses of the individual drugs or improved quality of life, compared to a monotherapy. A further benefit is that lower doses of the active ingredients of the combination of the invention can be used, for example, that the dosages need not only often be smaller but are also applied less frequently, or can be used in order to diminish the incidence of side-effects. This is in accordance with the desires and requirements of the patients to be treated.

With respect to the combinations according to the present invention as described hereinbefore and hereinafter they may be used for simultaneous use or sequential use in any order, e.g. for separate use or as a fixed combination.

The combinations according to the present invention comprises a “kit of parts” in the sense that both agents a and b can be dosed independently or by use of different fixed combinations with distinguished amounts of the components at different time points. The parts of the “kit of parts” can then e.g. be administered simultaneously or chronologically staggered, that is at different time points and with equal or different time intervals for any part of the “kit of parts”. Preferably, the time intervals are chosen such that the effect on the treated disease or condition in the combined use of the parts is larger than the effect that would be obtained by use of only any one of the components.

The effective dosage of each of the combination partners employed in the combination of the invention may vary depending on the particular compound or pharmaceutical composition employed, the mode of administration, the condition being treated, the severity of the condition being treated. Thus, the dosage regimen of the combination of the invention is selected in accordance with a variety of factors including the route of administration. A physician, clinician or veterinarian of ordinary skill can readily determine and prescribe the effective amount of the single active ingredients required to alleviate, counter or arrest the progress of the condition. Optimal precision in achieving concentration of the active ingredients within the range that yields efficacy without toxicity requires a regimen based on the kinetics of the active ingredients' availability to target sites.

Daily dosages for agent a) or agent b) or will, of course, vary depending on a variety of factors, for example the compound chosen, the particular condition to be treated and the desired effect. In general, however, satisfactory results are achieved on administration of agent a) at daily dosage rates of the order of about 0.1 to about 100 mg/kg per day, as a single dose or in divided doses.

In case of PPAR agonist an approximate daily dose of from about 1 mg to about 360 mg is to be estimated, preferably a daily dose of from 1 mg to 100 mg, more preferably a daily dose of from 1 mg to 50 mg, e.g. for a patient of approximately 75 kg in weight.

In case of ACE inhibitors, preferred dosage unit forms of ACE inhibitors are, for example, tablets or capsules comprising e.g. from about 5 mg to about 20 mg, preferably 5 mg, 10 mg, 20 mg or 40 mg, of benazepril; from about 6.5 mg to 100 mg, preferably 6.25 mg, 12.5 mg, 25 mg, 50 mg, 75 mg or 100 mg, of captopril; from about 2.5 mg to about 20 mg, preferably 2.5 mg, 5 mg, 10 mg or 20 mg, of enalapril; from about 10 mg to about 20 mg, preferably 10 mg or 20 mg, of fosinopril; from about 2.5 mg to about 4 mg, preferably 2 mg or 4 mg, of perindopril; from about 5 mg to about 20 mg, preferably 5 mg, 10 mg or 20 mg, of quinapril; or from about 1.25 mg to about 5 mg, preferably 1.25 mg, 2.5 mg, or 5 mg, of ramipril. Preferred is t.i.d. administration.

A preferred combination is the combination of Compound A, preferably in form of acetate salt, with an ACE inhibitor, e.g. as disclosed above.

1. Compound 3-(1.H.-indol-3-yl)-4-[2-(4-methyl-piperazin-1-yl)-quinazolin-4-yl]-pyrrole-2,5-dione, 3-(1H.-indol-3-yl)-[2-(piperazin-1-yl)-quinazolin-4-yl]-pyrrole-2,5-dione, 3-[3-(4,7-Diaza-spiro[2.5]oct-7-yl)-isoquinolin-1-yl]-4-(7-methyl-1H-indol-3-yl)-pyrrole-2,5-dione or a pharmaceutically acceptable salt thereof for use in the preparation of a medicament for treating, preventing or delaying diabetic complications.

2. The compound for use according to claim 1 wherein the complication is nephropathy, neuropathy or cardiomyopathy.

3. The compound for use according to claims 1 or 2 wherein the compound is the acetate salt of 3-(1.H.-indol-3-yl)-4-[2-(4-methyl-piperazin-1-yl)-quinazolin-4-yl]-pyrrole-2,5-dione.

4. A method of treating or preventing diabetic nephropathy, neuropathy or cardiomyopathy, or delaying their progression, comprising administering an effective amount of 3-(1.H.-indol-3-yl)-4-[2-(4-methyl-piperazin-1-yl)-quinazolin-4-yl]-pyrrole-2,5-dione, 3-(1.H.-indol-3-yl)-4-[2-(piperazin-1-yl)-quinazolin-4-yl]-pyrrole-2,5-dione or a pharmaceutically acceptable salt thereof to a subject in need of such treatment.

5. A method of treating or preventing diabetic cardiomyopathy, or delaying its progression, comprising administering an effective amount of 3-[3-(4,7-Diaza-spiro[2.5]oct-7-yl)-isoquinolin-1-yl]-4-(7-methyl-1H-indol-3-yl)-pyrrole-2,5-dione or a pharmaceutically acceptable salt thereof.

6. A pharmaceutical composition for treating or preventing diabetic complications comprising 3-(1.H.-indol-3-yl)-4-[2-(4-methyl-piperazin-1-yl)-quinazolin-4-yl]-pyrrole-2,5-dione, 3-(1.H.-indol-3-yl)-[2-(piperazin-1-yl)-quinazolin-4-yl]-pyrrole-2,5-dione, 3-[3-(4,7-Diaza-spiro[2.5]oct-7-yl)-isoquinolin-1-yl]-4-(7-methyl-1H-indol-3-yl)-pyrrole-2,5-dione or a pharmaceutically acceptable salt thereof, together with one or more pharmaceutically acceptable diluents or carriers therefore, wherein the complication is nephropathy, neuropathy or cardiomyopathy.

7. A pharmaceutical combination comprising a) a first agent which is selected from 3-(1H.-indol-3-yl)-4-[2-(4-methyl-piperazin-1-yl)-quinazolin-4-yl]-pyrrole-2,5-dione, 3-(1H.-indol-3-yl)-4-[2-(piperazin-1-yl)-quinazolin-4-yl]-pyrrole-2,5-dione, and pharmaceutically acceptable salt thereof, and b) at least one active ingredient selected from the group consisting of an HDL increasing compound, a PPAR delta compound, a non-glitazone type PPARγ agonist, an anti-hypertensive agent, a cholesterol absorption modulator, an apo-A1 analog or mimetics, a renin inhibitor, a thrombin inhibitor, an aldosterone inhibitor, a GLP-1 agonist, a glucagon receptor antagonist, a cannabinoid receptor 1 antagonist, an anti-obesity agent, an inhibitor of platelet aggregation, a PPAR agonist, a prosclerotic growth factor, or a pharmaceutically acceptable salt thereof.

8. A combination according to claim 6 wherein the first agent is 3-(1.H.-indol-3-yl)-4-[2-(4-methyl-piperazin-1-yl)-quinazolin-4-yl]-pyrrole-2,5-dione or the acetate salt thereof.

9. A pharmaceutical combination comprising 3-[3-(4,7-Diaza-spiro[2.5]oct-7-yl)-isoquinolin-1-yl]-4-(7-methyl-1H-indol-3-yl)-pyrrole-2,5-dione or a pharmaceutically acceptable salt thereof, and at least one active ingredient selected from the group consisting of an HDL increasing compound, an anti-hypertensive agent, a cholesterol absorption modulator, an apo-A1 analog or mimetics, a renin inhibitor, a thrombin inhibitor, an aldosterone inhibitor, a glucagon receptor antagonist, a cannabinoid receptor 1 antagonist, an anti-obesity agent, an inhibitor of platelet aggregation, a PPAR agonist, a prosclerotic growth factor, or a pharmaceutically acceptable salt thereof.

10. A method of treating or preventing diabetes or diabetic complications or delaying their progression comprising co-administration, e.g. concomitantly or in sequence, of a therapeutically effective amount of a 3-(1.H.-indol-3-yl)-[2-(4-methyl-piperazin-1-yl)-quinazolin-4-yl]-pyrrole-2,5-dione, 3-(1.H.-indol-3-yl)-4-[2-(piperazin-1-yl)-quinazolin-4-yl]-pyrrole-2,5-dione, and pharmaceutically acceptable salt thereof, and a second drug substance, to a subject in need of such treatment, wherein the second drug substance is selected from the group consisting of HDL increasing compounds, anti-diabetics, an anti-hypertensive agent, cholesterol absorption modulator, apo-A1 analogs and mimetics, renin inhibitors, thrombin inhibitors, aldosterone inhibitors, GLP-1 agonists, glucagon receptor antagonists, cannabinoid receptor 1 antagonists, anti-obesity agents; inhibitors of platelet aggregation, PPAR agonist, DPP-IV inhibitor, prosclerotic growth factor, or a pharmaceutically acceptable salt thereof.
